(19) United States
(12) Patent Application Publication
(10) Pub. No.: US 2008/0221383 A1
(43) Pub. Date: Sep. 11, 2008

(54) TISSUE EXCISION DEVICES AND METHODS

(75) Inventors: Bryce Way, San Jose, CA (US); Donald F. Schomer, Englewood, CO (US); Minh Tran, Westminster, CA (US); Alberto Cantu, San Francisco, CA (US); Paul M. Sand, San Carlos, CA (US); Herbert H. Mertens, Escondido, CA (US)

Correspondence Address: CONLEY ROSE, P.C.;David A. Rose, P. O. BOX 3267, HOUSTON, TX, 77253-3267, US

(73) Assignee: VERTOS MEDICAL, INC., San Jose, CA (US)

(21) Appl. No.: 12/029,816

(22) Filed: Feb. 12, 2008

Related U.S. Application Data
(60) Provisional application No. 60/889,367, filed on Feb. 12, 2007.
(60) Provisional application No. 61/015,588, filed on Dec. 20, 2007.

(51) Int. Cl. A61B 17/285; A61N 5/01

(52) U.S. Cl. 600/3; 606/114

(57) ABSTRACT

A tissue excision device comprises a handle. In addition, the tissue excision device comprises an elongate tissue capture member extending from the handle. The tissue capture member has a longitudinal axis and comprises a free end distal the handle. Further, the free end of the tissue capture member includes a tip, a tissue capture recess, and at least one slot extending through the free end in the tissue capture recess. Still further, the tissue excision device comprises an elongate tubular cutting member coupled to the handle. The cutting member slidingly and coaxially receives the tissue capture member. In addition, the cutting member has a free end distal the handle that includes a cutting.

### CROSS-REFERENCE TO RELATED APPLICATIONS

This application claims benefit of U.S. provisional application Ser. No. 60/889,367 filed Feb. 12, 2007, and entitled “Percutaneous Bone and Tissue Shearing Device,” which is hereby incorporated herein by reference in its entirety. This application also claims benefit of U.S. provisional application Ser. No. 61/015,588 filed Dec. 20, 2007, and entitled “Tissue and Bone Excision Devices and Methods of Using the Same,” which is hereby incorporated herein by reference in its entirety.

### STATEMENT REGARDING FEDERALLY SPONSORED RESEARCH OR DEVELOPMENT

Not Applicable.

### BACKGROUND

1. Field of the Invention

The present invention relates to devices and methods for treating spinal disorders using imaging guidance. More particularly, this invention also relates to devices and minimally invasive methods to relieve pressure on compressed nerves by shearing bone and/or tissue to increase the cross-sectional area available of the spinal canal and/or neural foramen.

2. Background of the Invention

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae with an intervertebral disc spacing adjacent vertebrae. The vertebrae are stabilized by muscles and ligaments that hold the vertebrae in place and limit the movements of the vertebrae.

Referring to FIGS. 1 and 2, each vertebra 10 includes a vertebral body 12 that supports a vertebral arch 14. A median plane MP generally divides vertebra 10 into two substantially equal lateral sides. Vertebral body 12 has the general shape of a short cylinder and is anterior to the vertebral arch 14. The vertebral arch 14 together with vertebral body 12 encloses a space termed the vertebral foramen 15. The succession of vertebral foramen 15 in adjacent vertebrae 10 along the vertebral column define the vertebral canal (spinal canal), which contains the spinal cord.

Vertebral arch 14 is formed by two pedicles 24 which project posteriorly to meet two laminae 16. The two laminae 16 meet posteriomedially to form the spinous process 18. At the junction of pedicles 24 and laminae 16, six processes arise. Two transverse processes 20 project posterolaterally, two superior articular processes 22 project generally superiorly and are positioned superior to two inferior articular processes 25 that generally project inferiorly. The superior articular processes 22 of each vertebra 10 are coupled to corresponding inferior articular processes 25 of the immediately superior vertebra 10 to form a facet joint complex 31.

Vertebral foramen 15 defines a generally oval or tri-oval shaped space that accommodates and protects spinal cord 28. Spinal cord 28 comprises a plurality of nerves 34 surrounded by cerebrospinal fluid (CSF) and an outermost sheath or membrane called the dural sac 32. The CSF filled dural sac 32 containing nerves 34 is relatively compressible. Within vertebral foramen 15 posterior to spinal cord 28 is the ligamentum flavum 26. Laminae 16 of adjacent vertebral arches 14 in the vertebral column are joined by the relatively broad, elastic ligamentum flavum 26.

Referring now to FIGS. 3 and 4, the spatial orientation and alignment of adjacent vertebrae 10 are maintained by a disc 29 disposed between each pair of adjacent vertebral bodies 12, facet joint complex 31, and the muscles and ligaments (e.g., ligamentum flavum 26) extending between adjacent vertebrae 10. A lateral opening to the spinal canal and vertebral foramen 15, referred to as a neural foramen 30, is positioned on either side of the vertebral column between adjacent vertebrae 10 and defined by the vertebral bodies 12, pedicles 24, superior articular processes 22, and inferior articular processes 25 of adjacent vertebrae 10. Nerve roots 35 extending from spinal cord 28 exit the vertebral column through neural foramen 30. The outside of nerve roots 35 comprise a protective sheath or sleeve.

In some degenerative conditions of the spine, stenosis or narrowing of the vertebral foramen 15 and/or neural foramen 30 can occur. Sufficient narrowing of the vertebral foramen 15 and/or neural foramen 30 may result in compression of dural sac 32, spinal cord, nerves 34, nerve roots 35, and blood vessels within the spinal canal and neural foramen. Symptoms associated with stenosis of the vertebral foramen and neural foramen 30 include lower back and leg pain, as well as weakness and numbness of the legs.

In general, spinal stenosis can arise from a variety of sources including thickening of the ligamentum flavum, subluxation, facet joint hypertrophy, osteophyte formation, underdevelopment of spinal canal, spondylosis deformans, degenerative intervertebral discs, degenerative spondylolisthesis, degenerative arthritis, excess fat in the epidural space, ossification of the vertebral accessory ligaments, genetics, gradual “wear and tear,” or combinations thereof. A less common cause of stenosis, which usually affects patients with morbid obesity or patients on oral corticosteroids, is excess fat in the epidural space. Spinal stenosis may also affect the cervical and, less commonly, the thoracic spine. Patients suffering from stenosis of the vertebral foramen 15 and/or neural foramen 30 are typically first treated with exercise therapy, analgesics, and anti-inflammatory medications. These conservative treatment options frequently fail. If symptoms are severe, surgery is required to decompress the nerves 34 in the spinal cord and/or nerves 34 extending through neural foramen 30.

Two common surgical procedures to treat narrowing of vertebral foramen 15 are a laminectomy and a laminotomy. As shown in FIG. 5, in a laminectomy, the posterior portion of vertebral arch 14 extending between lamina 16 is completely removed. As shown in FIG. 6, in a laminotomy, a portion of one lamina 16 of vertebral arch 14 is removed. In FIG. 6, the inferior portion of lamina 16 of a superior vertebra 10 is removed and the superior portion of the corresponding lamina 16 of an immediately inferior vertebra 10 is removed. Both procedures (laminectomy and laminotomy) are intended to treat stenosis of vertebral foramen 15 by widening vertebral foramen 15 to at least partially decompressing spinal cord 28 and nerves 34 passing therethrough.

Two common surgical procedures to treat narrowing of neural foramen 30 are a facetecomy and foraminotomy. As shown in FIG. 7, a facetecomy is the partial or complete removal of the facet joint complex 31 defining the narrowed neural foramen 30. As shown in FIG. 8, a foraminotomy is the partial removal or modification of one or more of the bony structures defining neural foramen 30 (i.e., modification of vertebral body 12, inferior pedicle 24, superior pedicle 24, superior articular processes 22, and/or inferior articular processes 25 defining the stenosed neural foramen 30). Both procedures (facetecomy and foraminotomy) are intended to treat stenosis of neural foramen 15 by widening neural foramen 15 to at least partially decompress nerve roots 35 extending therethrough. It should be appreciated that a facetecomy may also be used to treat stenosis of the vertebral foramen 15.

Conventionally, access to the vertebra to perform a laminectomy, laminotomy, facetecomy, or foraminotomy is achieved by making an incision the back, stripping the muscles and supporting structures away from the spine, thereby exposing the posterior aspect of the vertebral column. Thus, such surgical procedures are typically performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently.

Much of the pain and disability after an open laminectomy, laminotomy, facetecomy or foraminotomy results from the tearing and cutting of the back muscles, blood vessels, supporting ligaments, and nerves that occurs during the exposure of the spinal column. Also, because the spine stabilizing back muscles and ligaments are stripped and detached from the spine during the laminectomy, these patients frequently develop spinal instability post-operatively.

Minimally invasive techniques offer the potential for less post-operative pain and faster recovery compared to traditional open surgery. Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures. However, it should be appreciated that because nerves 34 pass through vertebral foramen 15 and neural foramen 30, any surgery, regardless of whether open or percutaneous, includes a risk of damage to the nerves of the spinal cord.

Accordingly, there remains needs in the art for methods, techniques, and devices for treating stenosis of the vertebral foramen and neural foramen, as well as for other spinal disorders. Such methods and devices would be particularly well received if they were minimally invasive, without requiring open surgery, and reduced the risk of damage to the dural sac and nerves.

### BRIEF SUMMARY OF SOME OF THE PREFERRED EMBODIMENTS

In accordance with at least one embodiment of the invention, a tissue excision device comprises a handle. In addition, the tissue excision device comprises an elongate tissue capture member extending from the handle. The tissue capture member has a longitudinal axis and comprises a free end distal the handle. Further, the free end of the tissue capture member includes a tip, a tissue capture recess, and at least one slot extending through the free end in the tissue capture recess. Still further, the tissue excision device comprises an elongate tubular cutting member coupled to the handle. The cutting member slidingly and coaxially receives the tissue capture member. Moreover, the cutting member has a free end distal the handle that includes a cutting.

In accordance with other embodiments of the invention, a method for treating stenosis of a neural foramen of a patient comprises visualizing the neural foramen. In addition, the method comprises outlining a nerve or nerve root in the region of interest with a contrast agent. Further, the method comprises percutaneously positioning a distal end of a portal proximal the neural foramen to be excised. Still further, the method comprises inserting a tissue excision device into a proximal end of the portal external the patient. Moreover, the method comprises advancing the tissue excision device through the portal to the neural foramen. In addition, the method comprises modifying the neural foramen with the tissue excision device.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a more complete understanding of the invention, reference is made to the accompanying drawings, wherein:

FIG. 1 is a partial cross-sectional view of the spine from the space between two adjacent vertebrae, showing the upper surface of one vertebra;

FIG. 2 is a view of the spine from the space between two adjacent vertebrae, showing the lower surface of a vertebra;

FIG. 3 is a perspective view of a pair of adjacent vertebrae;

FIG. 4 is a partial side view of the vertebral column;

FIG. 5 is a posterior view of the spine schematically illustrating a laminectomy;

FIG. 6 is a posterior view of the spine schematically illustration a laminotomy;

FIG. 7 is a posterior view of the spine schematically illustrating a facetecomy;

FIG. 8 is a lateral side view of the spine schematically illustrating a foraminotomy;

FIG. 9 is a side view of an embodiment of a tissue excision device in an opened position;

FIG. 10 is a cross-sectional view of the tissue excision device of FIG. 9;

FIG. 11 is a side view of the tissue excision device of FIG. 9 in the closed position;

FIG. 12 is an enlarged partial cross-sectional view of the handle of the tissue excision device of FIG. 9;

FIG. 13 is an enlarged cross-sectional view of the distal end of the tissue excision device of FIG. 9;

FIG. 14 is an enlarged top view of the distal end of the tissue excision device of FIG. 9;

FIGS. 15-18 are alternative embodiments of the distal end of the tissue capture member of FIG. 9;

FIGS. 19-21 are selected schematic partial cross-sectional views of a laminectomy or laminotomy employing the tissue excision device of FIG. 9; and

FIGS. 22-26 are selected schematic views of a foraminotomy employing the tissue excision device of FIG. 9.

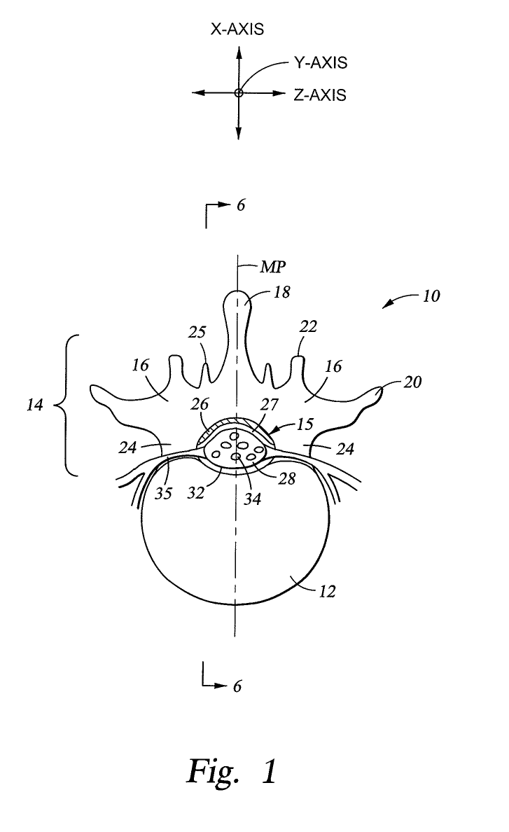

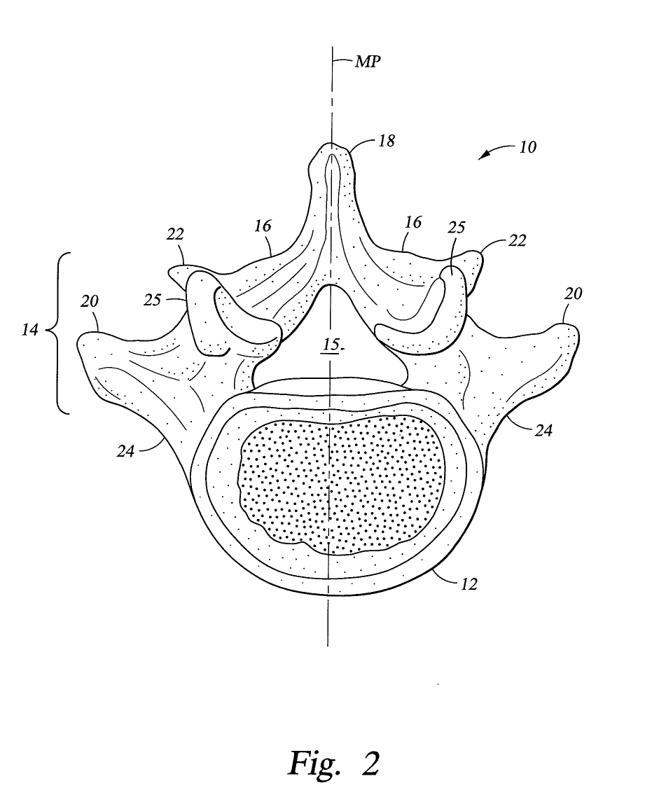

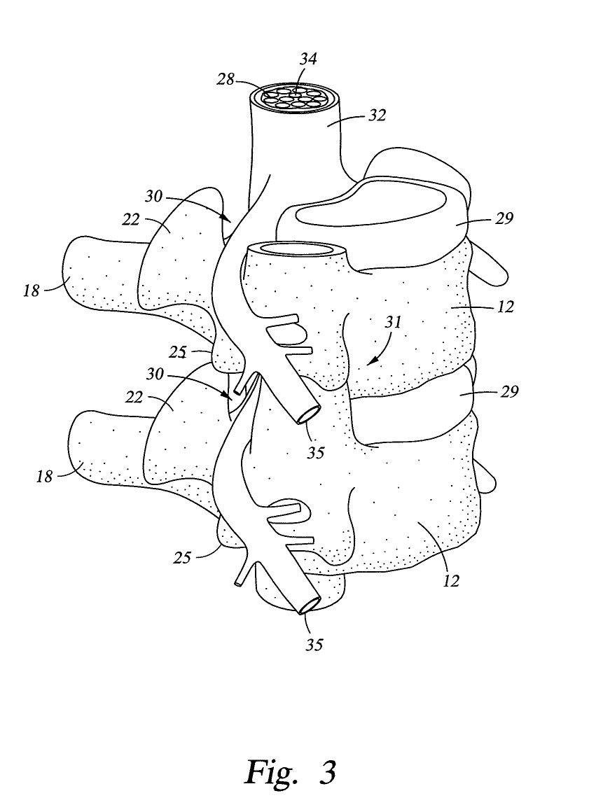

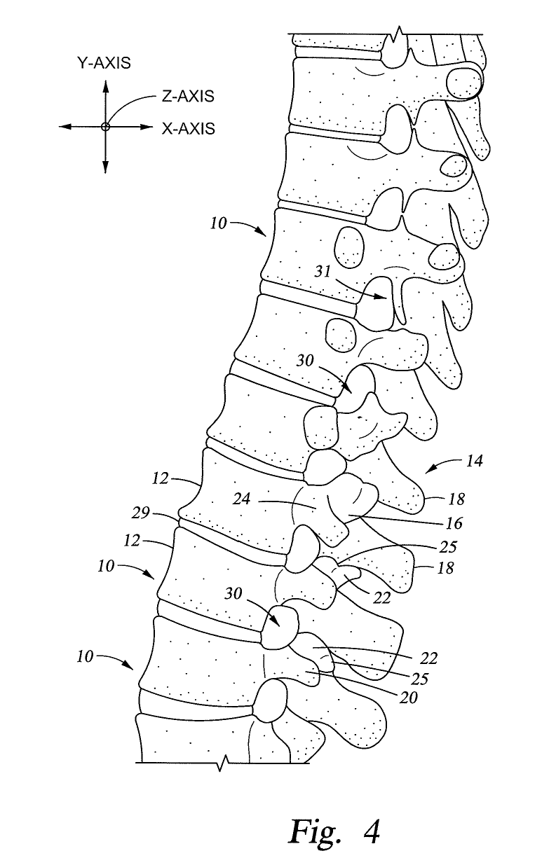

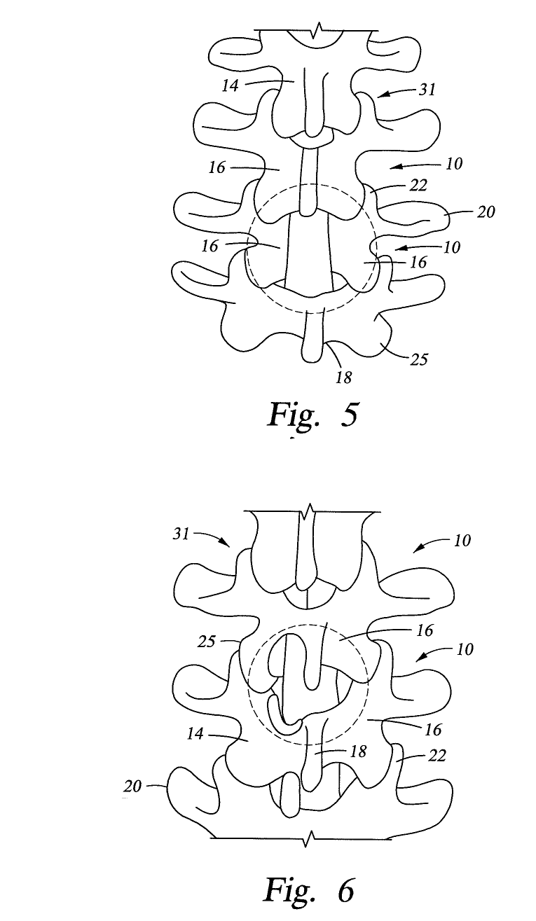

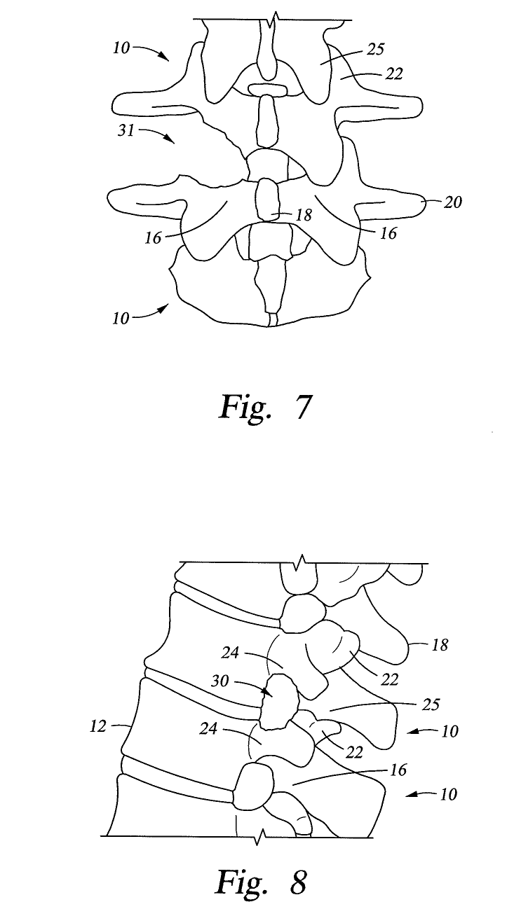

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

For purposes of this discussion, the x-, y-, and z-axes are shown in several figures to aid in understanding the descriptions that follow. The x-, y-, and z-axes have been assigned as follows. The x-axis is perpendicular to the longitudinal axis of the vertebral column and perpendicular to the coronal/frontal plane (i.e., x-axis defines anterior vs. posterior relationships). The y-axis runs generally parallel to the vertebral column and perpendicular to the transverse plane (i.e., y-axis defines superior vs. inferior relationships). The z-axis is perpendicular to the longitudinal axis of the vertebral column and perpendicular to the median/midsagittal plane (i.e., z-axis defines the lateral right and left sides). The set of coordinate axes (x-, y-, and z-axes) are consistently maintained throughout although different views of vertebrae and the spinal column may be presented.

It is to be understood that the median or midsagittal plane passes from the top to the bottom of the body and separates the left and the right sides of the body, and the spine, into substantially equal halves (e.g., two substantially equal lateral sides). Further, it is to be understood that the frontal/coronal plane essentially separates the body into the forward (anterior) half and the back (posterior) half, and is perpendicular to the median plane. Still further, it is to be understood that the transverse plane is perpendicular to both the median plane and coronal plane and is the plane which divides the body into an upper and a lower half.

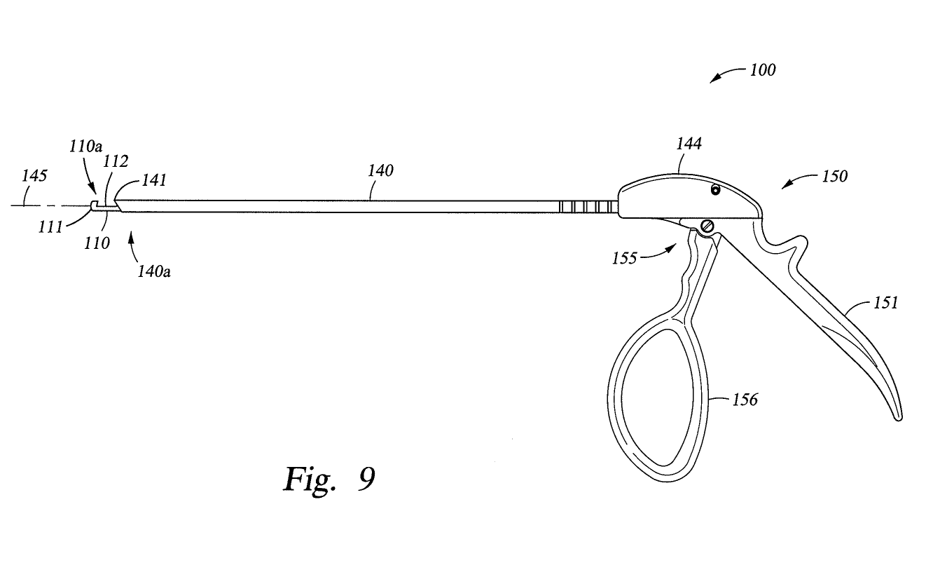

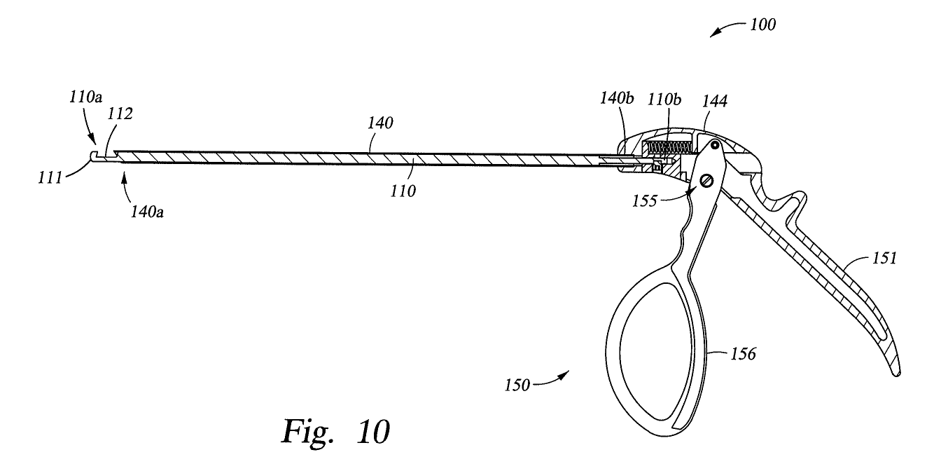

Referring to FIGS. 9 and 10, an embodiment of a tissue excision instrument or device 100 is shown. In general, tissue excision device 100 may be used in any open spinal procedure, image guided procedure, minimally invasive procedure, percutaneous surgery, or combinations thereof, but is specifically designed to cut and remove tissue to perform a laminectomy, laminotomy, facetecomy, or foraminotomy. In general, the tissue that may excised by device 100 includes, without limitation, bone, bone dentin, cartilage, ligaments, disc material, fat, muscle, and/or other soft tissues.

Tissue excision device 100 comprises an elongate tissue capture member 110, an elongate tubular cutting member 140 that slidingly receives tissue capture member 110, and a handle 150 coupled to members 110, 140. Tissue capture member 110 and cutting member 140 slide axially relative to each other upon actuation of handle 150.

Handle 150 includes a base arm 151 and a lever arm 156 pivotally connected at a pivot joint 155 along their lengths. In this embodiment, lever arm 156 is pivotally connected to base arm 151 with a pin that passes through aligned bore in arms 151, 156. Thus, arms 151, 156 may be rotated relative to each other about pivot joint 155. During use of device 100, base arm 151 is held in the palm of the user's hand and lever arm 156 is grasped by the fingers of the users same hand.

Referring still to FIGS. 9 and 10, tissue capture member 110 includes a free or distal end 110a and a handle end 110b coupled handle 150. More specifically, handle end 110b is fixed to base arm 151 such that tissue capture member 110 does not move translationally or rotationally relative to base arm 151. In this embodiment, handle end 110b is fixed to base arm 151 with a set screw. In addition, free end 110a includes a tip 111 and a tissue capture recess 112 adapted to receive tissue to be cut and removed.

Tissue capture recess 112 includes a distal shoulder 112a, a proximal shoulder 112b, and a lower surface 112c extending therebetween. Distal shoulder 112a is oriented at an angle μ relative to lower surface 112c. In this embodiment, angle μ is between 0° and 90°, and more specifically about 60°. Orienting distal shoulder 112a at an angle μ is between 0° and 90° offers the potential to improve the ability of tissue capture recess 112 to grasp and retain tissue extending into tissue capture recess 112. In other embodiments, the distal shoulder (e.g., distal shoulder 112a) is oriented at an angle μ between 90° and 180°.

Tubular cutting member 140 has a longitudinal axis 145 and co-axially receives tissue capture member 110. Thus, tubular cutting member 140 and tissue capture member 110 share the same longitudinal axis 140. Cutting member 140 includes a free or distal end 140a and a handle end 140b coupled to handle 150 with a cover 144. Distal end 140a includes a cutting edge 141 adapted to slide axially across tissue capture recess 112 and shear any tissue extending from tissue capture recess 112. As used herein, the term “axially” may be used to describe positions or movement along or parallel to longitudinal axis 145, whereas the term “radially” may be used to describe positions or movement perpendicular to longitudinal axis 145.

In this embodiment, members 110, 140 are generally cylindrical, each having a circular cross-section taken perpendicular to longitudinal axis 145. The outer radius of member 110 is the same or slightly less than the inner radius of member 140, such that member 110 may be coaxially disposed within member 140. In addition, with the exception of distal end 110a including tissue capture recess 112, the outer radius of each member 110, 140 is uniform along its respective length. In general, tissue capture member 110 and tissue cutting member 140 may have any suitable cross-sectional geometry (e.g., rectangular, oval, etc.) and size (radius, width, length, etc.). However, to enable insertion and advancement of members 110, 140 into a cylindrical access cannula or portal conventionally used for percutaneous surgeries, members 110, 140 each preferably have a circular cross-section taken perpendicular to longitudinal axis 145.

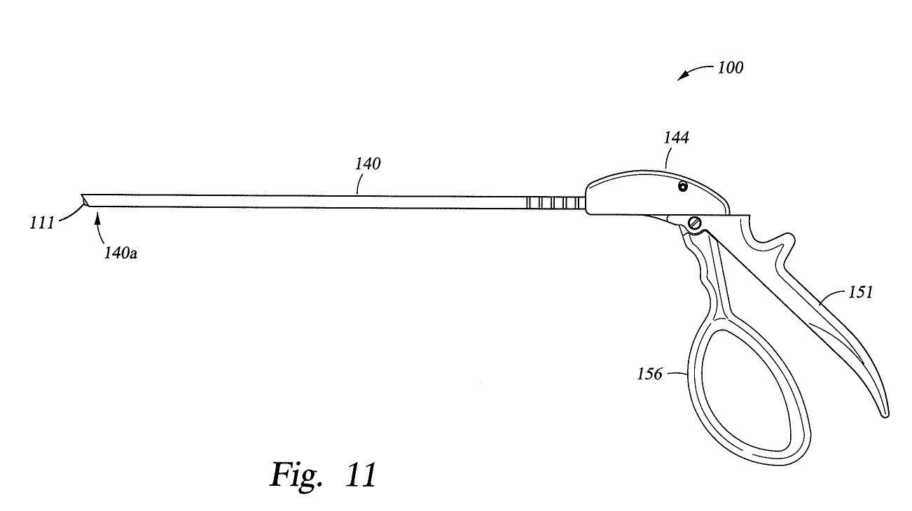

Referring now to FIGS. 9 and 11, device 100 and cutting member 140 may generally be described as having an open position (FIG. 9) in which distal end 140a does not extend axially across tissue capture recess 112, and a closed position (FIG. 11) in which distal end 140a extends completely axially across tissue capture recess 112. When device 100 is in the open position, tissue capture recess 112 is completely open to receive tissue, and when device 100 is in the closed position, tissue capture recess 112 is completely closed off by distal end 140a. When device 100 is transitioned to the closed position, any tissue disposed within tissue capture recess 112 is cut or sheared by cutting edge 114 as it slides across tissue capture recess 112. Thus, device 100 may be described as removing tissue by a shearing action as opposed to a crushing action common with most conventional rongeurs. Without being limited by this or any particular theory, as compared to the removal of tissue by a crushing action, the sequential shearing and removal of superficial layers of bone and tissues by shearing can incrementally widen an orifice (e.g., neural foramen, vertebral foramen, etc.) with a reduced amount of damage to adjacent structures. For the removal of bone and/or tissues of the spine (e.g., foraminotomy, laminotomy, etc.), decreased collateral damage and injury offers the potential to reduce postoperative mechanical instability that can produce postoperative complications, delayed patient symptoms, and delayed patient recovery. It should be appreciated that device 100 has a plurality of intermediate portions between the open position and the closed position in which distal end 140a extends partially across tissue capture recess 112.

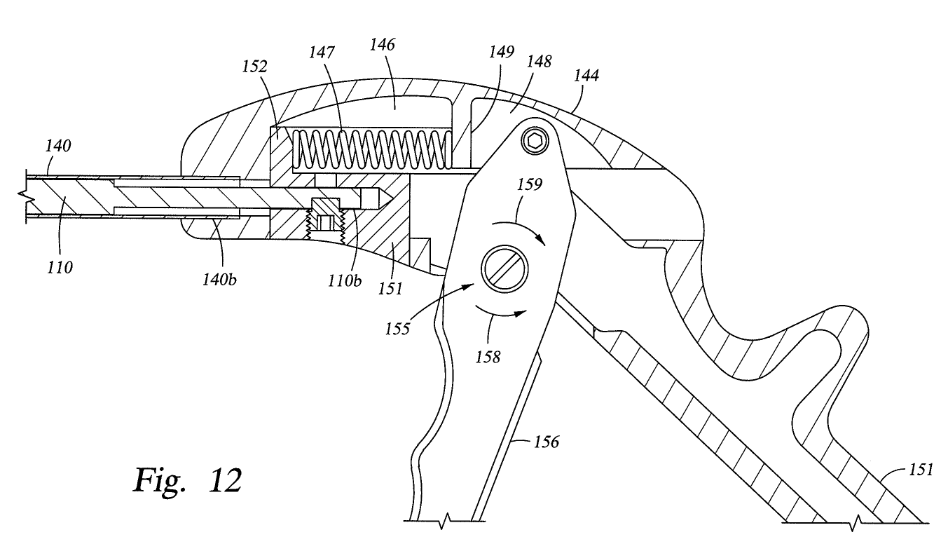

Referring now to FIGS. 10 and 12, handle end 140b is fixed to cover 144 and cover 144 is coupled to the upper portion of base arm 151 and the upper end of lever arm 156. In particular, cover 144 slidingly engages base arm 151 such that cover 144, and hence cutting member 140, is free to move axially relative to base arm 151, but is restricted from moving rotationally or laterally relative to base arm 151. In this embodiment, the bottom of cover 144 includes a first recess 146 and a second recess 148 divided by a wall 149.

The upper end of lever arm 156 extends into second recess 148 and is pivotally coupled to cover 144. In particular, cover 144 includes an internal pin 147 that extends laterally across second recess 148. Pin 147 passes through a bore 157 in the upper end of lever arm 156. Rotation of lever arm 156 about pivot joint 155 toward base arm 156 in direction 158 results in the axial movement of cover 144 and cutting member 140 to the left, thereby closing device 100 (FIG. 11). However, rotation of lever arm 156 about pivot joint 155 away from base arm 156 in direction 159 results in the axial movement of cover 144 and cutting member to the right, thereby opening device 100 (FIG. 9).

Referring still to FIGS. 10 and 12, device 100 is biased to the open position (FIG. 9) by a biasing member 147. In particular, base member 156 includes an extension 152 extending upward into first recess 146. Biasing member 147 is axially positioned between extension 152 and wall 149, and urges extension 152 and wall 149 apart, thereby biasing device 100 to the open position. However, with sufficient force applied to lever arm 156, the user of device 100 can overcome the biasing forces generated by biasing member 147 and transition device 100 to the closed position. In this embodiment, biasing member 147 is a spring, however, in general, biasing member may comprise any suitable device capable of biasing device 100 to the open position.

It should be appreciated that biasing member 147 is disposed within first recess 146, and thus, is not visible from the outside of device 100. In this sense, biasing member 147 may be referred to as an “internal” biasing member. Since biasing member 147 is disposed within first recess 146, there is less risk of biasing member 147 interfering or inhibiting use of device 100. In some conventional surgical tools, a leaf spring is externally disposed in conjunction with the handle of the device (e.g., externally between the arms of the handle). During use of such conventional devices, the external leaf spring may interfere with the user's hand and fingers that grasp the handle and actuate the device. For instance, the users hand may get pinched in the external leaf spring. However, embodiments described herein include an internal biasing member 147 which offers the potential to reduce the likelihood of interfering with the use of device 100.

Referring now to FIGS. 9 and 11, during use, device 100 is placed in the open position. Then device 100 is oriented and positioned such that the tissue (e.g., bone, cartilage, soft tissue, etc.) to be cut extends into tissue capture recess 112. Then, the user actuates handle 150, thereby transitioning device 100 to the closed position. As cutting edge 141 slides across tissue capture recess 112, the tissue extending into recess 112 is sheared by cutting edge 141 and captured in recess 112.

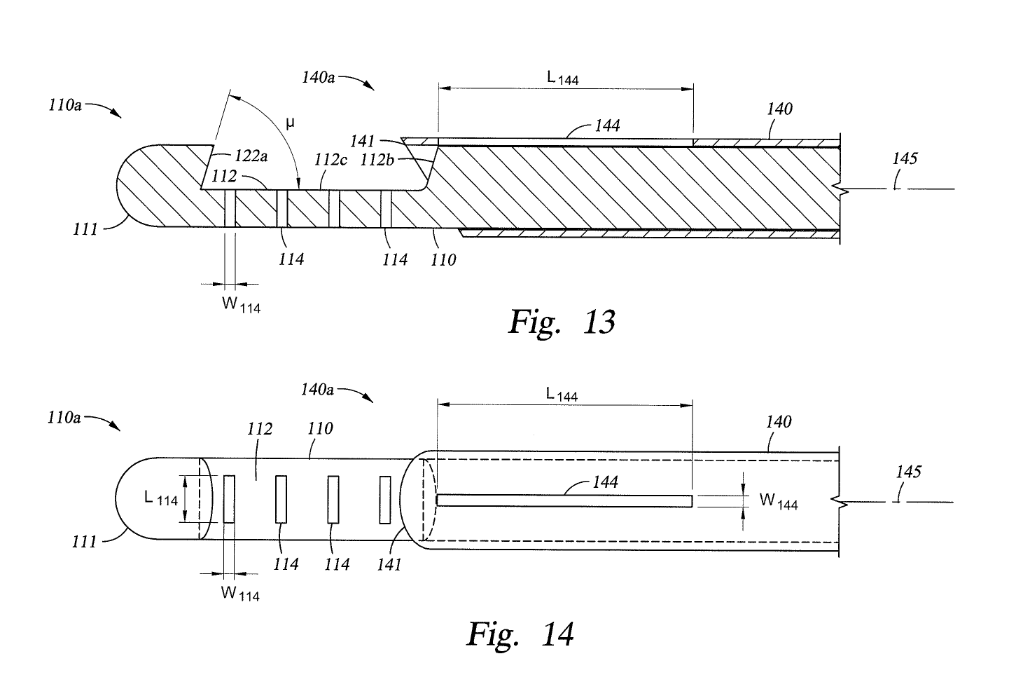

Referring now to FIGS. 13 and 14, in this embodiment, tip 111 of tissue capture member 110 is generally smooth and spherical or dome-shaped. In general, a smooth and blunt tip (e.g. rounded, spherical, etc.) is preferred to minimize the risk of inadvertently cutting or damaging the dural sac or nerves while treating stenosis of the vertebral foramen and/or neural foramen. Such geometries offer the potential to contact and gently urge sensitive nerves and/or dural sac during surgery without cutting or damaging the nerves, nerve roots and/or dural sac. Although a smooth, blunted distal tip is preferred for surgeries proximal sensitive nerves and/or dural sac, in other embodiments, the tip (e.g., tip 111) of the tissue capture member (e.g., tissue capture member 110) may have other geometries.

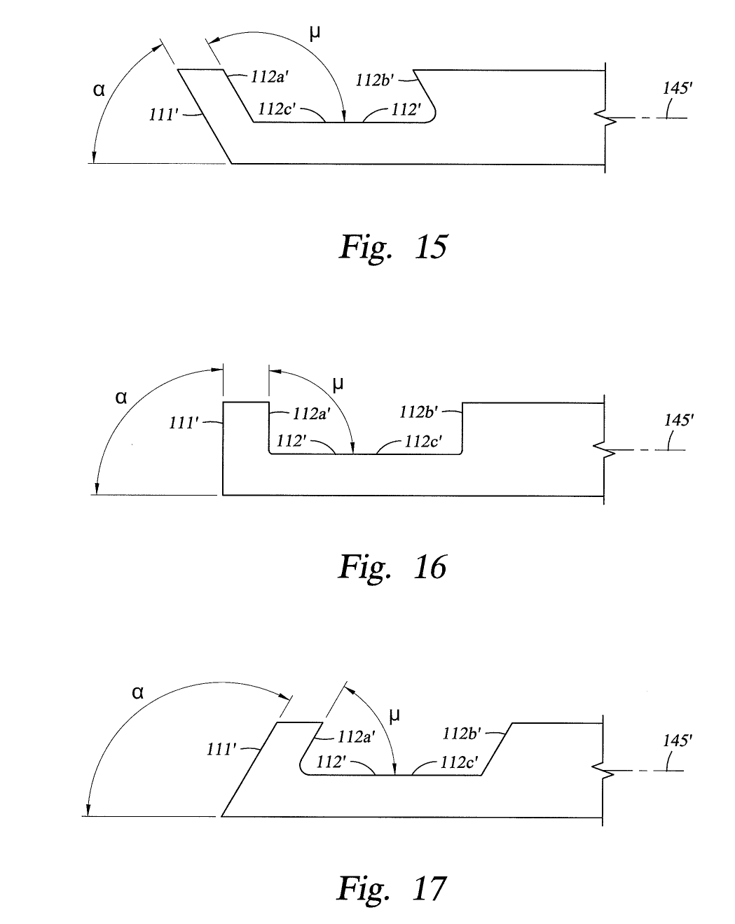

Referring now to FIGS. 15-18, alternative embodiments for the distal tip (e.g., distal tip 110a) of the tissue capture member (e.g., tissue capture member 110) are shown. In FIG. 15, the distal tip 111′ of the tissue capture member is generally planar and is oriented at an angle α relative to the longitudinal axis 145′ between 0° and 90°. In this embodiment, angle α is about 60°. In addition, the distal shoulder 112a′ of tissue capture recess 112′ is oriented at an angle μ relative to lower surface 112c′ between 90° and 180°, and more specifically about 120°. In FIG. 16, the distal tip 111′ is generally planar and is oriented at an angle α of about 90°. In addition, the distal shoulder 112a′ of tissue capture recess 112′ is oriented at an angle μ of about 90°. In FIG. 17, the distal tip 111′ is generally planar and is oriented at an angle α between 90° and 180°, and more specifically, about 120°. In addition, the distal shoulder 112a′ of tissue capture recess 112′ is oriented at an angle μ of about 60°. Without being limited by this or any particular theory, a tip 111″ angled relative to the longitudinal axis 145′ offers the potential for improved fluoroscopic visualization by projecting the tip beyond any shadowing from the handle and proximal shaft of the device.

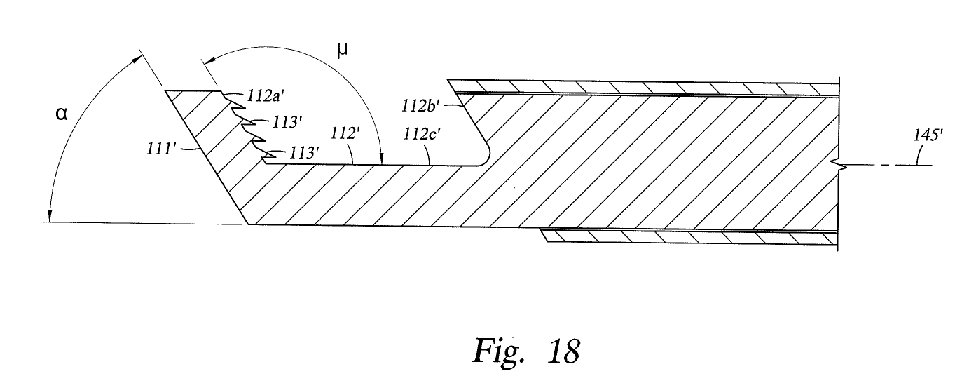

In some embodiments, the distal shoulder (e.g., distal shoulder 112a) of the tissue capture recess (e.g., tissue capture recess 112) may include teeth, serrations, or barbs to grasp tissue extending into the tissue capture recess. For instance, referring to FIG. 18, the distal shoulder 112a′ of tissue capture recess 112′ comprises tissue grasping teeth or serrations 113′ angled back to grasp tissue extending into tissue capture recess 112′. Teeth or serrations 113′ may be particularly useful on embodiments where distal shoulder 112a′ is oriented at an angle μ greater than or equal to 90°. Although teeth or serrations 113′ are shown on the distal shoulder 112a′ in this embodiment, in general, tissue grasping teeth or serrations may be provided on any suitable area of the tissue capture recess 112′ including, without limitation, distal shoulder 112a′, proximal shoulder 112b′, lower surface 112c′, or combinations thereof.

Referring again to FIGS. 13 and 14, distal end 110a of tissue capture member 110 includes a plurality of slots 114 extending completely through distal end 110a within tissue capture recess 112. In this embodiment, each slot 114 is elongate and rectangular, and further, are oriented perpendicular to central axis 145 in side view (FIG. 13) and top view (FIG. 14). Each slot 114 has a width W₁₁₄ measured parallel to central axis 145 and a length L₁₁₄ measured perpendicular to central axis 145 in top view. In this embodiment, each slot 114 has substantially the same geometry and dimensions. However, in other embodiments, one or more of the slots (e.g., slots 114) may have a different geometry and/or dimensions. For instance, a single slot tracking back and forth the distal end (e.g., distal end 110a) generally perpendicular to the central axis (e.g., axis 145) and having a wavy or “snakelike” shape in top view may be used.

In addition, distal end 140a of tissue cutting member 140 includes a slot 144 extending through its upper side. In this embodiment, slot 144 is elongate and rectangular, and further, is oriented parallel to central axis 145 in side view (FIG. 13) and top view (FIG. 14). Slot 144 has a width W₁₄₄ measured perpendicular to central axis 145 in top view (FIG. 14) and a length L₁₄₄ measured parallel to central axis 145. In other embodiments, the slot in the tissue cutting member (e.g., slots 144 of tissue cutting member 140) may have a different geometry and/or dimensions. Further, in some embodiments, more than one slot (e.g., slot 144) may be provided in the tissue cutting member (e.g., tissue cutting member 140). As best shown in FIG. 14, slot 144 is generally perpendicular to slots 114.

It should be appreciated that during percutaneous, non-invasive surgical procedures direct visualization of the surgical tools and devices disposed in the patient is not available. Rather, visualization is achieved through the use of x-ray or fluoroscopic technologies (e.g., digital fluoroscopy). To increase the likelihood of success of the surgery and to minimize inadvertent damage to sensitive tissues (e.g., nerves) proximal the surgical site, it is preferred that the surgeon maintain three-dimensional spatial orientation of the surgical tools and devices extending into the patient. Due to the geometries necessitated by patient positioning for percutaneous spinal surgery, the likely orientations of the fluoroscopic equipment, and the geometries of conventional rongeurs, it is typically difficult to visualize the open and closed jaws of most conventional rongeurs under fluoroscopy. However, inclusion of slots 114 in tissue capture member 110 offer the potential to enhance the fluoroscopic visualization of the distal end of device 100 and the surgeon's spatial awareness of the distal end of device 100. As a result, slots 114 offer the potential to improve the accuracy and precision with which the surgeon can position the distal end of device 100. In particular, under fluoroscopic visualization, the absence of material in slots 114 increases the contrast, and hence visibility, of slots 114 relative to the remainder of distal end 110a of tissue capture member 110. As a result, slots 114 offer the potential to improve the accuracy and precision with which the surgeon can position the distal end of device 100.

Similarly, inclusion of slot 144 in tissue cutting member 140 offers the potential to enhance the fluoroscopic visualization of the distal end 140a. In particular, under fluoroscopic visualization, the absence of material in slot 144 increases the contrast, and hence visibility, of slot 144 relative to the remainder of distal end 140a of tissue cutting member 140. However, since tissue capture member 110 is coaxially disposed with tubular tissue cutting member 140 beneath slot 144, the degree of contrast and fluoroscopic visualization of slot 144 relative to the remainder of distal end 140a may be slightly reduced as compared to the contrast and fluoroscopic visualization of slots 114 relative to the remainder of distal end 110a. As distal end 110a typically leads device 100 into the patient, visualization of distal end 110 is particularly preferred.

Although slots 114, 144 are shown and described as passing completely through distal ends 110a, 140a, in other embodiments, one or more of the slots (e.g., slots 114, slot 144)) may extend to a particular depth, but not pass completely through the material. Without being limited by this or any particular theory, the reduced material will result in increase fluoroscopic contrast. However, the deeper the slots and the greater the absence of material, the greater the contrast under fluoroscopic imaging.

Improved visualization of distal end 110a, and to a lesser extend improved visualization of distal end 140a, offer the potential to enhance axial and radial positioning of the distal end of device 100. With the distal end of device 100 sufficiently positioned proximal the tissue to be excised, the surgeon may rotate device 100 about longitudinal axis 145 with handle 150 to circumferentially orient the tissue capture recess 112 in the proper position to engage the tissue to be excised. The positioning of slots 114 in tissue capture recess 112 offers the potential to improve the surgeon's particular positioning of tissue capture recess 112.

Referring still to FIGS. 13 and 14, slots 114, and to a lesser extent slot 144, also offer the potential to enhance the surgeon's spatial awareness of cutting edge 141 relative to tissue capture recess 112. In other words, slots 114 and slot 144 may enable the surgeon to determine when device 100 is open (i.e., tissue cutting member 140 does not extend across tissue capture recess 112), closed (i.e., tissue cutting member 140 extends completely across tissue capture recess 112), or in an intermediate position (i.e., tissue cutting member 140 extends partially across tissue capture recess 112). For instance, when device 100 is in the open position, none of slots 114 are covered by cutting member 140, and hence, should be visible under fluoroscopy. As cutting edge 141 slides axially across tissue capture recess 112, one or more slots 114 will become covered by cutting member 140 and less visible under fluoroscopy. By including a predetermined number of slots 114 and/or a predetermined axial spacing between adjacent slots 114, the surgeon may be able to assess the degree of closure of device 100. For example, if there are four evenly spaced slots (e.g., slots 114) in the tissue capture recess (e.g., tissue capture recess 112), clear visibility of the two distal slots and reduced or no visibility of the two proximal slots would indicate that the tissue excision device (e.g., device 100) is about half way closed. Moreover, as previously described, slots 114 are perpendicular to slot 144 in top view. Consequently, as device 100 is transition between the open and closed positions, slots 114, 144 will cross under fluoroscopic visualization to form an “X” or “T”.

The components of device 100 (e.g., base arm 151, lever arm 156, cover 144, tissue capture member 110, tissue cutting member 140, etc.) may comprise any suitable materials including, without limitation, metals, metal alloys, non-metals, composites, or combinations thereof. The components of device 100 are preferably made from biocompatible materials. For instance, handle 260 and lever 250 may be machined or molded from plastic or metal such as 400 series stainless steel (SS), 17 series SS, and 300 series SS, or NiTi. Since members 110, 140 are advanced into the patient, engage and cut tissue, and may be advanced through tissue, members 110, 140 preferably comprise rigid biocompatible materials such as 400 series SS, 17 series SS, and 300 series SS, or NiTi.

In some embodiments, one or more components of device 100 may be made from a polymer or ceramic that is relatively lightweight and biocompatible. Further, polymeric and ceramic materials are both X-ray, fluoroscopic, MRI, and CT compatible and can enhance visualization if either of these modalities is utilized for image guidance. Such embodiments may be particularly suited to single use designs of device 100. For instance, handle 150 may comprise a polymer discarded after a single use. As another example, tissue capture member 110 and/or tubular cutting member 140 may comprise a polymer that is discarded after a single use. As still one more example, to ensure a single use device 100, pivot joint 155 may comprises a polymeric hinge pin that deforms during steam sterilization.

The various components of device 100 may be machined, cast, molded, laser cut, EMD, etc. In some embodiments, electro polishing is used to sharpen certain parts, such as cutting edge 211 of second member 210. Surface treatments such as diamond knurl, sand blasting, bead blasting, media blasting, plasma etching, etc. may also be used. For assembly, the components may be coupled by any suitable means including, without limitation, press fitting, gluing, welding, swaging, riveting, screwing, bolting, and the like.

It should be appreciated that percutaneous fluoroscopically guided procedures require optimal orientation of the X-ray source and image capture device (e.g. image intensifier) relative to the anatomic structures being treated. In the case of the cutting device, the X-ray source is preferably oriented perpendicularly to the cutting surface for near optimal visualization. However, in many applications this preferred orientation is not possible due to the anatomic constraints required by the patient's anatomy. Thus, embodiments described herein offer the potential to enhance spatial awareness and fluoroscopic control by insuring visualization of the relative position (open or closed) of the cutting surface from one or more fluoroscopic angles. Although the following procedures are described in terms of fluoroscopic visualization, alternatively, the operating physician may elect to perform these procedures with imaging guidance using magnetic resonance imaging (MRI) or computed tomography (CT). For such embodiments, the tools and devices (e.g., tissue excision device 100) may be constructed from MRI or CT compatible materials to optimize visualization within these environments.

Moreover, embodiments of the procedures and methods described below assume common and typical orientations of the anatomical structures of interest in the patient. For patients with anatomical structures having atypical orientations, embodiments of the procedure may be adjusted as appropriate to account for such differences.

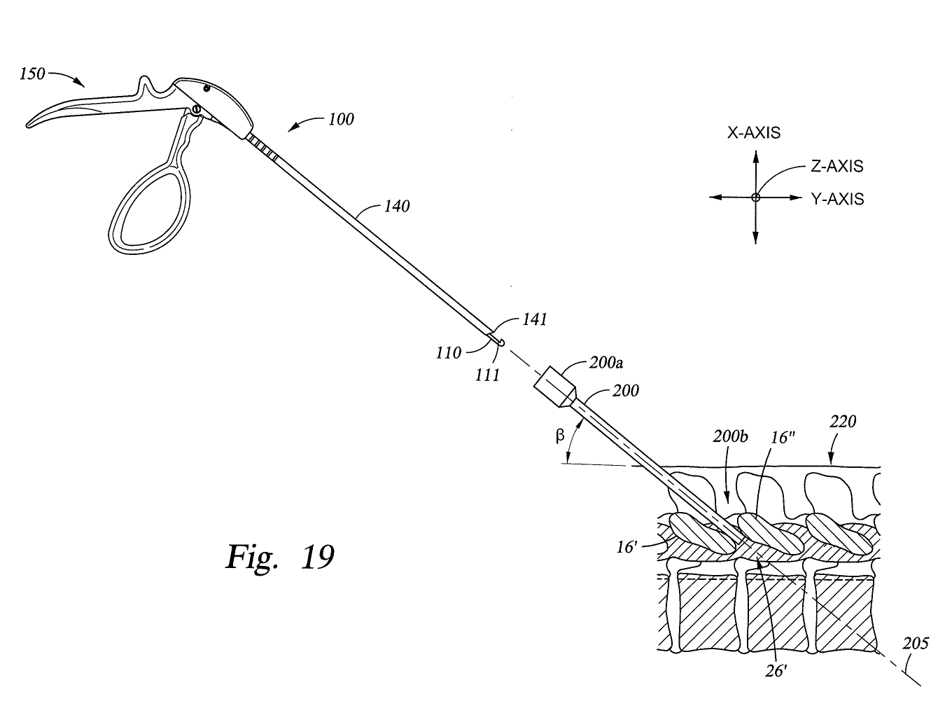

Referring now to FIGS. 19-21, selected views of a percutaneous laminectomy or laminotomy employing tissue excision device 100 are shown. Referring first to FIG. 19, the patient is placed in a prone position amenable to fluoroscopic imaging of the portion of the spine to be treated. In particular, for a laminotomy or laminectomy, the imaging system is oriented to maximize visualization of the lamina to be modified during the laminotomy or laminectomy. In most cases, an anterior-posterior (AP) view of the spine. As used herein, the phrase “anterior-posterior” view may be used to describe an imaging view generally perpendicular to the dorsal skin surface. Since the dorsal skin surface is generally parallel to the frontal plane dividing the body into a front half and back half, the “anterior-posterior” view may also be described as perpendicular to the frontal plane. One or more additional fluoroscopic views (e.g., lateral side view or lateral-oblique view) may be employed to visualize the depth of the tissue excision device 100. Then, an elongate access cannula or portal 200 having a longitudinal axis 205, a receiving end 200a, and a distal end 200b is positioned to provide percutaneous access to an inferior lamina 16′, a superior lamina 16″, and the ligamentum flavum 26′ extending therebetween. The long axes of laminae 16′, 16″ are typically oriented at an angle between 45° and 90° relative to the dorsal skin surface 220, and more specifically at an angle between 60° to 75° relative to the dorsal skin surface 220. Thus, to access the interlaminar space (i.e., space between laminae 16′, 16″), portal 200 is preferably oriented with its longitudinal axis 205 at a caudal-cranial angle β relative to the dorsal skin surface 220 between about 5° and 90°, and more preferably between 60° and 75°. As used herein, the phrase “caudal-cranial angle” may be used to describe an angle measured in the median or midsagittal plane (i.e., in the x-y plane) relative to the dorsal skin surface. Since the dorsal skin surface is generally parallel to the frontal plane dividing the body into a front half and back half, the caudal-cranial angle may also be described as an angle measured in the median or midsagittal plane (i.e., in the x-y plane) relative to the frontal plane. In addition, portal 200 is preferably oriented with its longitudinal axis 205 at a lateral-oblique angle between 5° and 60° relative to the transverse plane, and more preferably between 30° and 45°. As used herein, the phrase “lateral-oblique angle” may be used to describe an angle measured in the frontal or coronal plane (i.e., angle measured in the y-z plane) relative to the transverse plane dividing the body into upper and lower halves. Portal 200 is axially advanced until distal end 200b is disposed between lamina 16′, 16″. Once sufficiently positioned, receiving end 200a is disposed external to the patient, distal end 200b is positioned adjacent lamina 16′, 16″ and ligamentum flavum 26′. Further, once sufficiently positioned, the orientation of portal 200 is preferably maintained for the remainder of the procedure.

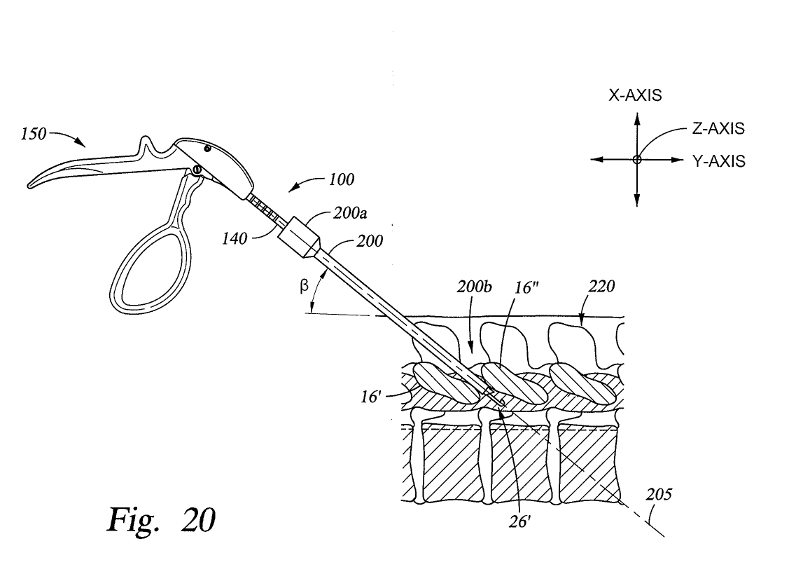

Moving now to FIG. 20, the distal end of device 100 is inserted into, and axially advanced through portal 200 to the region of interest. With device 100 configured in the open position with tissue capture recess 112 exposed and cutting edge 141 withdrawn, tissue capture recess 112 is positioned immediately inferior to superior lamina 16″ with an inferior portion of superior lamina 16″ extending into tissue capture recess 112. As previously described, device 100 and tissue capture recess 112 may be positioned and oriented under fluoroscopic visualization and with the aid of slots 114.

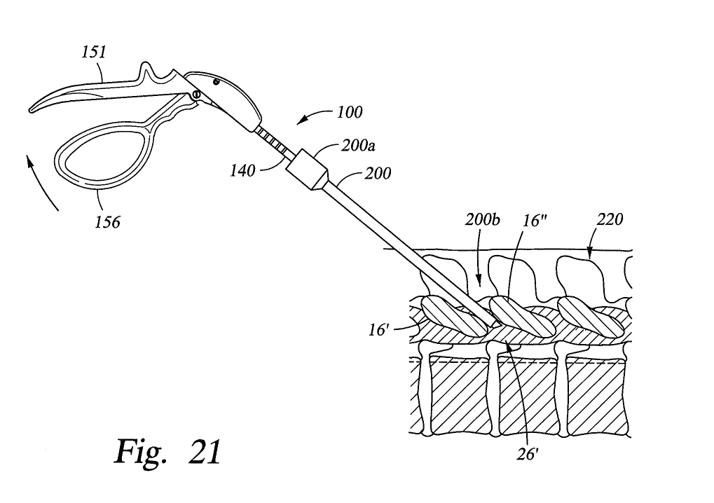

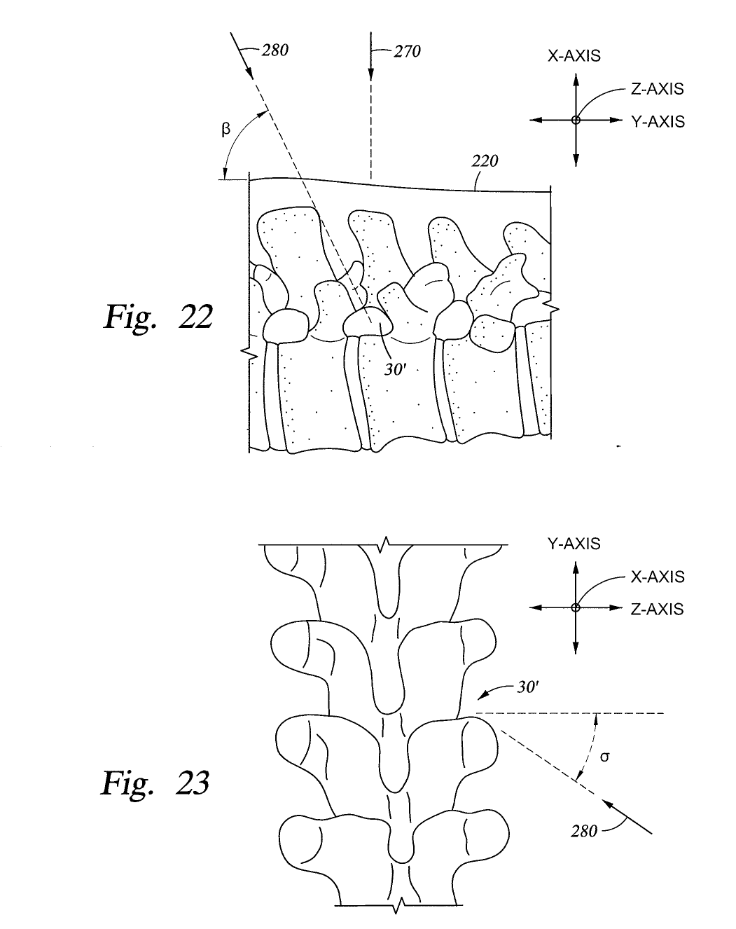

Moving now to FIG. 21, device 100 is actuated by squeezing lever arm 156 towards base arm 151, thereby axially advancing tissue cutting member 140 relative to tissue capture member 200 and moving cutting edge 141 across tissue capture recess 112. As cutting edge 141 moves across tissue capture recess 112, the inferior portion of superior lamina 16″ disposed within tissue capture recess 112 is sheared off with cutting edge 141 and captured in recess 112. Tissue excision device 100 may then be withdrawn from portal 200 and opened to remove the excised bone and tissue within tissue capture recess 112, and the process repeated to remove more bone and tissue to decompress the spinal cord. In some embodiments, the tissue capture member (e.g., tissue capture member 110) may be a tubular including a plunger slidingly disposed the tissue capture member. Such a plunger may be axially advanced into the tissue capture recess (e.g., tissue capture recess 112) to expel excised tissue therefrom. This general process is preferably repeated until sufficient bone and tissue are removed to reduce stenosis. In other embodiments, a passage or bore providing percutaneous access to the tissue capture recess (e.g., tissue capture recess 112) may be provided through the tissue capture member (e.g., tissue capture member 110) and the handle (e.g., handle 150). In such embodiments, bone and tissue excision may be repeated without withdrawing the tissue excision device (e.g., device 100) from the access portal (e.g., portal 200). For instance, a wire having a barb may be advanced through the passage in the handle and the tissue capture member to the tissue capture recess. Any bone or tissue within the tissue capture recess may be grasped by the barb and withdrawn through the passage in the tissue capture member and the handle. As another example, suction may be utilized to remove tissue from the tissue capture recess of the tissue capture recess.

The procedure described with respect to FIGS. 19-21 may also be used to excise portions of ligamentum flavum 26′ in the interlaminar space between laminae 16′, 16″. For such a procedure, the distal end of device 100 is preferably contoured and shaped to fit specifically under the laminae (e.g., laminae 16′, 16″) and joint facets (e.g., joint facet complex 31) to enhance excision of such tissues and bone under fluoroscopic image guided surgeries. In particular, device 100 is configured in the open position and tissue capture recess 112 is positioned such that a portion of ligamentum flavum 26′ extends into tissue capture recess 112. Then, device 100 is transitioned to the closed position with handle 150. As cutting edge 141 slides across tissue capture recess 112, the portion of ligamentum flavum 26′ disposed within tissue capture recess 112 is sheared off and captured in recess 112. The excised ligamentum flavum 26″ tissue in tissue capture recess 112 may be removed by any of the means previously described.

Referring now to FIGS. 22-26, selected views of a percutaneous foraminotomy employing tissue excision device 100 are shown. Beginning with FIGS. 22 and 23, the patient is placed in a prone position amenable to fluoroscopic imaging of the portion of the spine to be treated. Identification of the neural foramen 30′ to be modified and positioning of the instruments (e.g., device 100) is preferably confirmed and maintained throughout the procedure with fluoroscopic guidance in at least two planes or views—the lateral-oblique view described below and the anterior-posterior (AP) view. Such fluoroscopic visualization and guidance offers the potential to verify and guide depth of entry into neural foramen 30′.

In the AP position, imaging 270 is oriented substantially perpendicular to the frontal or coronal plane (i.e., perpendicular to the y-z plane and perpendicular to the patient's dorsal skin surface). As best shown in FIGS. 22 and 23, for most cases, in the lateral-oblique position, imaging 280 is oriented at a caudal-cranial angle β relative to the dorsal skin surface 220 between about 5° and 30°, and more preferably between about 10° and 15°, and at a lateral-oblique angle σ between about 15° and 60°, and more preferably between about 30° and 45°.

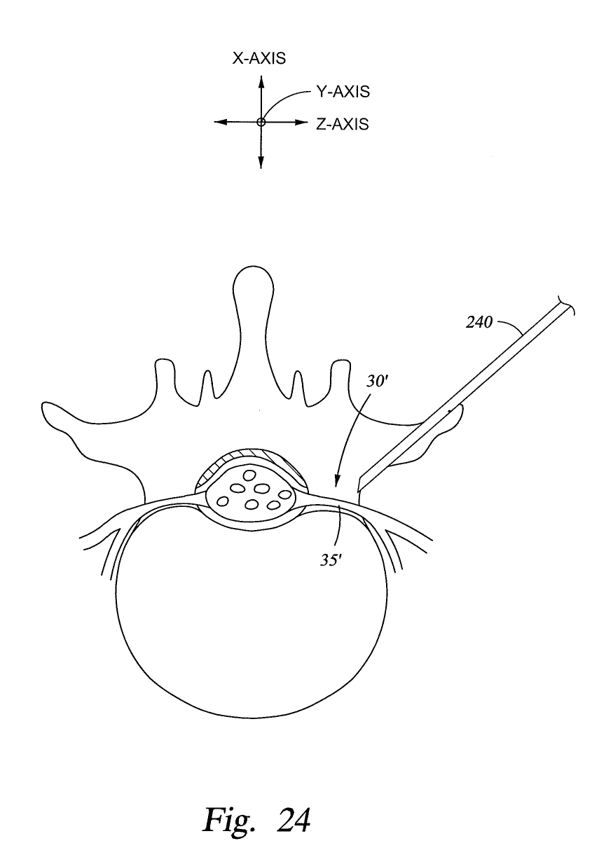

Referring now to FIG. 24, a spinal needle 240 (e.g., 22 gage or smaller spinal needle) is then advanced into the neural foramen 30′. The longitudinal axis of the spinal needle is preferably oriented at a caudal-cranial angle β relative to the dorsal skin surface 220 between about 5° and 30°, and more preferably between about 10° and 15°, and at a lateral-oblique angle σ between about 15° and 60°, and more preferably between about 30° and 45°, so that the neural foramen 30′ is visualized en-face relative to the X-ray source and image capture system. With this en-face visualization of neural foramen 30′, spinal needle 240 may be advanced along this trajectory towards neural foramen 30′. Depth of penetration of spinal needle 240 may be confirmed in the AP plane as defined by the X-ray source/image capture system. Utilizing spinal needle 240, exiting nerve root 35′ and its sleeve are outlined by a contrast agent. In general, any suitable contrast agent may be employed. After injecting the contrast agent, spinal needle 240 is withdrawn.

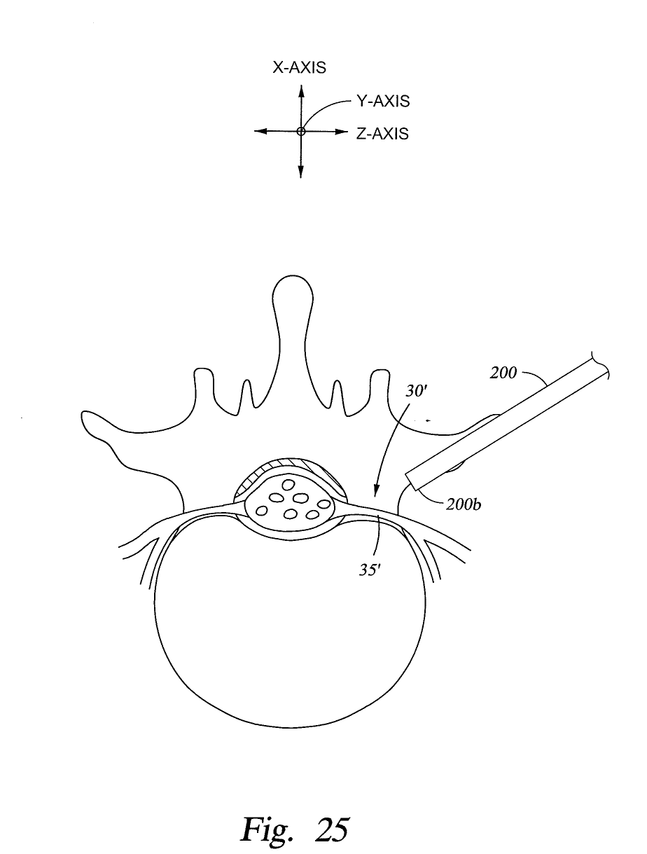

Referring now to FIG. 25, an elongate access cannula or portal 200 as previously described is inserted and advanced along a similar trajectory as spinal needle 240. With the aid of the fluoroscopic image guidance, access portal 200 is advanced until its distal tip 200b is positioned proximal to the opacified nerve root 35′ and associated sleeve.

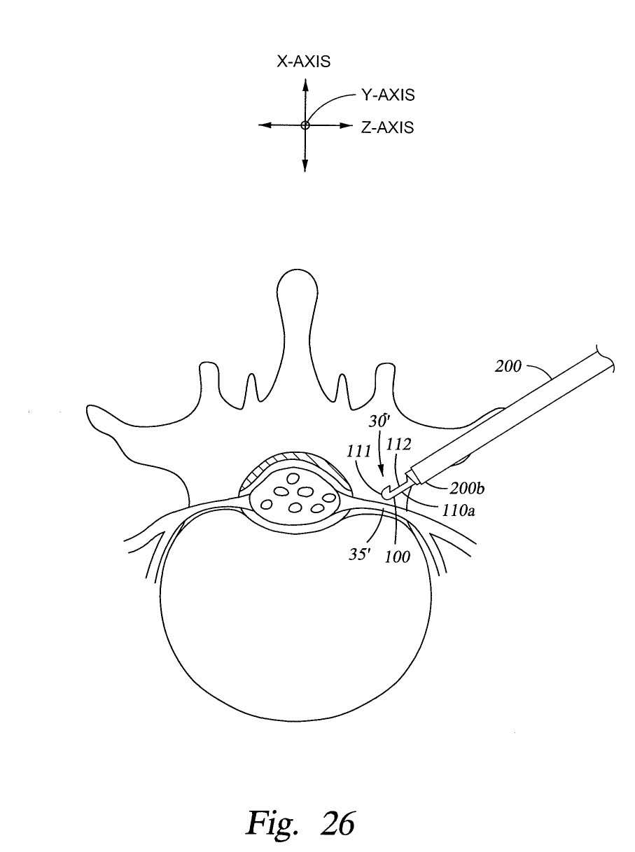

Referring now to FIG. 26, with access portal 200 sufficiently positioned, tissue excision device 100 is inserted into receiving end 200a of access portal 200 and advanced toward neural foramen 30′. With fluoroscopic guidance, distal tip 110a of device 100 is positioned such that the tissue to be excised (e.g., portions of the vertebral body, pedicles, superior articular processes, or inferior articular processes) extends into tissue capture recess 112. Positioning of tissue capture recess 112 may be aided by visualization of slots 114. Device 100 is then actuated by squeezing lever arm 156 towards base arm 151, thereby axially advancing tissue cutting member 140 relative to tissue capture member 200 and moving cutting edge 141 across tissue capture recess 112. As cutting edge 141 moves across tissue capture recess 112, the tissue disposed within tissue capture recess 112 is sheared off with cutting edge 141 and captured in recess 112. Tissue excision device 100 may then be withdrawn from portal 200 and opened to remove the excised bone and tissue within tissue capture recess 112, or percutaneously emptied as previously described. The process repeated to remove more bone and tissue to decompress nerve root 35.

Although embodiments of device 100 have been described for use in treating stenosis of the vertebral foramen and/or neural foramen, embodiments of device 100 may also be used to excise other bones or tissues, and further may be used in other methods such as the MILD method disclosed in U.S. patent application Ser. No. 11/193,581, which is hereby incorporated herein by reference in its entirety, or in the ILAMP method disclosed in U.S. patent application Ser. No. 11/382,349, which is hereby incorporated herein by reference in its entirety.

While preferred embodiments of this invention have been shown and described, modifications thereof can be made by one skilled in the art without departing from the scope or teaching of this invention. Accordingly, the invention is not limited to the embodiments described herein, but is only limited by the claims that follow, the scope of which shall include all equivalents of the subject matter of the claims. Likewise, the sequential recitation of steps in a claim, unless explicitly so stated, is not intended to require that the steps be performed in any particular order or that a particular step be completed before commencement of another step.

What is claimed is:

1. A tissue excision device comprising:
a handle;
an elongate tissue capture member extending from the handle, wherein the tissue capture member has a longitudinal axis and comprises a free end distal the handle;
wherein the free end of the tissue capture member includes a tip, a tissue capture recess, and at least one slot extending through the free end in the tissue capture recess;
an elongate tubular cutting member coupled to the handle, wherein the cutting member slidingly and coaxially receives the tissue capture member;
wherein the cutting member has a free end distal the handle that includes a cutting.

2. The device of claim 1 wherein the cutting member has an open position with the free end of the cutting member axially withdrawn from the tissue capture recess, and a closed position with the free end of the cutting member extending across the tissue capture recess.

3. The device of claim 2 wherein the cutting edge is adapted to shear tissue extending into the tissue capture recess when the cutting member is transitioned from the open position to the closed position.

4. The device of claim 1 wherein the at least one slot is elongate and is oriented perpendicular to the longitudinal axis of the tissue capture member.

5. The device of claim 4, wherein the at least one slot is rectangular.

6. The device of claim 1 wherein the free end of the tissue capture member comprises a plurality of slots extending through the free end in the tissue capture recess.

7. The device of claim 6 wherein each slot in the tissue capture member is elongate and is oriented perpendicular to the longitudinal axis of the tissue capture member.

8. The device of claim 7 wherein the slots are uniformly axially spaced apart.

9. The device of claim 1 wherein the tip of the tissue capture member is dome-shaped.

10. The device of claim 4 wherein the free end of the tissue cutting member comprises at least one slot extending through the upper portion of the free end.

11. The device of claim 10 wherein the at least one slot in the free end of the tissue cutting member is elongate and is oriented perpendicular to the at least one slot in the tissue capture member.

12. The device of claim 2 wherein the handle includes an internal spring that biases the cutting member to the open position.

13. A method for treating stenosis of a neural foramen of a patient comprising:
a) visualizing the neural foramen;
b) outlining a nerve or nerve root in the region of interest with a contrast agent;
c) percutaneously positioning a distal end of a portal proximal the neural foramen to be excised;
d) inserting a tissue excision device into a proximal end of the portal external the patient;
c) advancing the tissue excision device through the portal to the neural foramen; and
d) modifying the neural foramen with the tissue excision device.

14. The method of claim 13 wherein visualizing the neural foramen comprises:
orienting a first fluoroscopic imaging line substantially perpendicular to the dorsal skin of the patient, and
orienting a second fluoroscopic imaging line at a caudal-cranial angle between 5° and 30° and at a lateral-oblique angle between about 15° and 60°

15. The method of claim 14 wherein the neural foramen is defined by a vertebral body, a pedicle, a superior articular process of an inferior vertebra, and an inferior articular process of a superior vertebra, and wherein modifying the neural foramen comprises excising tissue from the vertebral body, the pedicle, the superior articular process of the inferior vertebra, or the inferior articular process of the superior vertebra.

16. The method of claim 15 wherein the tissue excision device comprises:
an elongate tissue capture member having a longitudinal axis and free end with a tissue capture recess;
an elongate tubular cutting member coaxially disposed about the elongate member, wherein the cutting member includes a free end having a cutting edge; and
wherein the cutting member has an open position with the free end axially withdrawn from the tissue capture recess, and a closed position with the free end extending across the tissue capture recess.

17. The method of claim 16 further comprising:
configuring the tissue excision device into the open position;
positioning a portion of the vertebral body, the pedicle, the superior articular process of the inferior vertebra, or the inferior articular process of the superior vertebra in the tissue capture recess;
transitioning the cutting member to the closed position; and
shearing the portion of the vertebral body, the pedicle, the superior articular process of the inferior vertebra, or the inferior articular process of the superior vertebra extending into the tissue capture recess.

18. The method of claim 16 wherein the free end of the tissue capture member includes at least one elongate slot disposed extending through the free end in the tissue capture recess.

19. The method of claim 18 wherein the at least one slot is oriented perpendicular to the longitudinal axis.

20. The method of claim 16 wherein the free end of the tissue capture member has a dome-shaped tip.

21. The method of claim 17 wherein the tissue cutting member includes an elongate slot in the upper portion of the free end, wherein the slot is oriented parallel to the longitudinal axis.
